(19) United States
(12) Patent Application Publication
(10) Pub. No.: US 2024/0252325 A1
(43) Pub. Date: Aug. 1, 2024

(54) VERTEBRAL DECOMPRESSION AND FUSION IMPLANT

(71) Applicant: Hunter MULLINS, Lexington, KY (US)

(72) Inventor: Hunter MULLINS, Lexington, KY (US)

(21) Appl. No.: 18/565,080

(22) PCT Filed: May 31, 2022
(86) PCT No.: PCT/US22/31662

§ 371 Date: Nov. 28, 2023

Related U.S. Application Data
(60) Provisional application No. 63/194,670, filed on May 28, 2021.

(51) Int. Cl. A61F 2/44; A61F 2/30; A61F 2/46

(52) U.S. Cl. CPC A61F 2/446; A61F 2/30767; A61F 2/4601; A61F 2/4611; A61F 2002/30537; A61F 2002/3092

(57) ABSTRACT

A novel and advantageous implant for vertebral decompression and fusion that disperses bone cement in and around the disc space to create support between and fusion of adjacent vertebral bodies is provided. The implant includes an interbody cage and an injection port. The interbody cage comprises a superior endplate, an inferior endplate, and a body between the superior endplate and the inferior endplate. The interbody cage includes one or more fenestrated openings to facilitate migration of injected bone cement out of the interbody cage and into the disc space. The injection port is configured for coupling to a bone cement injector such that bone cement may be injected through the injection port into the body with the coupling being able to withstand the force required to inject relatively viscous bone cement. The injection port may include a seal for sealing the injection port.

### FIELD OF THE INVENTION

The present disclosure relates to a novel and advantageous implant for vertebral decompression and fusion that is not dependent upon new bone growth. In particular, the present disclosure relates to a novel and advantageous implant for vertebral decompression and fusion that disperses bone cement in and around the disc space to create support between and fusion of adjacent vertebral bodies.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Various spinal fusion surgeries wherein adjacent vertebrae are fused are common. In general, this comprises removing a disc, replacing the disc with a cage, putting biologic graft material in the cage, and promoting bony ingrowth into the cage to get bony fusion. The bony ingrowth takes a significant amount of time (weeks to months) and the cage needs to be fixed in place during this time, typically using fixation elements such as bone screws. The fixation elements cause further trauma to the site of the spinal fusion. An additional complication is that, in some patients, bony fusion is unlikely to occur to the degree needed—due to osteoporosis, age, etc. There is a need for a device for spinal fusion that does not require extended periods of time for bony fusion to occur before the device is fixed in place.

More detailed description will now be given to conditions that lead to spinal fusion and procedures used for spinal fusion. Arthritis and degeneration (wear and tear) of the spine leads to a loss of normal spinal alignment and instability (abnormal movement), both of which may cause back pain and compression of the nerves. Nerves in the spine are naturally compressed and impinged with age. Compression of the nerve roots and narrowing of the spinal canal can be painful and can cause pain in the legs, such as in the calves, when walking, numbness and weakness in the legs, and bowel and/or bladder issues. Vertebral fusion decompression procedures can be done to offload pressure from the nerves.

Posterior lumbar decompression surgery typically involves removal of structures that are compressing the nerve root. These structures can include part of the lamina or the whole lamina, ligaments, and/or new bone (osteophytes). The lamina is the bony portion of the vertebra that rests behind the spinal cord. The lamina may be removed to access the spinal cord and nerves. Removal of part of the lamina is referred to as laminotomy. Removal of all of the lamina is referred to as laminectomy. Lumbar decompression surgery is effective in relieving the leg pain but the weakness, numbness, and the pins and needles effect in the legs (if present) may take months to resolve.

Spinal fusion, also called spondylodesis or spondylosyndesis, is a neurosurgical or orthopedic surgical technique that joins two or more vertebrae. This procedure can be performed at any level in the spine and substantially prevents movement between the fused vertebrae. The goal of this procedure is to decompress and create stability in the spine that eliminates motion and pain. If stability is not achieved, pain and discomfort continue. Spinal fusion involves removing the disc (which typically has extruded into the canal or a nerve root) and inserting an interbody cage into the vertebrae, with such cage being fixed in place by screws and the like. The screws are typically rods made of either titanium or stainless steel and are connected by rods and bone grafts around the vertebrae. The aim of the surgery is to prevent movement between the involved vertebrae and to realign the spinal column. This will reduce pain of nerve compression.

Current spinal fusion techniques involve a decompression of the effected stenotic levels of the lumbar and thoracic spine with the clinical goal of decompressing the levels of the spine and inserting an interbody cage to replace the removed disc. The interbody cage acts as a support for the fused, or joined, vertebral bodies of the spine. The interbody cage also acts as a conduit for inserting biologic bone graft forming elements that assist in forming bone scaffolding, with the long term goal of promoting fusion of at least the two adjacent levels with bony ingrowth. Pedicle screws are typically inserted at the affected levels and are designed to provide temporary fixation and stability of the levels of the spine to allow permanent stability and fusion. Current practice is fusion through bone growth, which is not predictable and takes a long time.

The overall construct involving the interbody cage, biologic bone growth, and pedicle screws yields collateral damage to the spine elements requiring healing. Further, the amount of time for fusion to occur is significant. In at least about 20-30% of patients fusion of the adjacent levels does not occur, which leads to further complications and costs. Even when the spinal fusion is successful, it often takes 4-6 months and up to a year for fusion to occur.

Thus, there is a need in the art for a surgical technique and implant that minimizes or eliminates the need for the pedicle screws and uncertainty of spine fusion.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

In one embodiment, a system for decompression and vertebral fusion is provided. The system includes a vertebral implant and bone cement. The vertebral implant comprises an interbody cage and an injection port. The interbody cage comprises a superior endplate, an inferior endplate, and a body between the superior endplate and the inferior endplate.

The injection port is configured for receiving a bone cement injector such that bone cement may be injected therethrough and into the interbody cage. The injection port may have a coupling mechanism for coupling to the bone cement injector. In one embodiment, the coupling mechanism may comprise threading within an inner cannula of the injection port. A seal may be provide for sealing the injection port such that injected bone cement cannot exit the injection port away form the interbody cage. The seal may comprise a one-way seal and/or an end cap.

The body of the interbody cage is configured to facilitate migration of injected bone cement out of the interbody cage, into disc space, and around the vertebral implant. In some embodiments, the body of the interbody cage may be defined by a wall extending at least partially between the superior endplate and the inferior endplate. The wall may include fenestrated openings for facilitating the migration of injected bone cement. In some embodiments, at least one of the superior endplate and the inferior endplate may also include fenestrated openings for facilitating the migration of injected bone cement. The bone cement may comprise a polymethyl methacrylate modified to have a low polymerization temperature and high porosity compare to unmodified polymethyl methacrylate.

In another embodiment, a vertebral implant for decompression and vertebral fusion is provided. The implant includes an interbody cage and an injection port. The interbody cage comprises a superior endplate, an inferior endplate, and a body between the superior endplate and the inferior endplate. The body is defined by a wall extending at least partially between the superior endplate and the inferior endplate. The wall includes a plurality of fenestrated openings. The injection port is configured for receiving a bone cement injector such that bone cement may be injected through the injection port into the body. The injection port may include a coupling mechanism for coupling to the bone cement injector/injection device. A seal may be provide for sealing the injection port such that injected bone cement cannot exit the injection port away from the interbody cage. The seal may comprise a one-way seal and/or an end cap.

The fenestrated openings are configured to facilitate migration of injected bone cement out of the interbody cage, into disc space, and around the vertebral implant. At least one of the superior endplate and the inferior endplate may include a plurality of fenestrated openings to facilitate the migration of injected bone out of the interbody cage, into disc space, and around the vertebral implant.

In some embodiments, the fenestrated openings are evenly distributed over the wall. In other embodiments, the fenestrated opening are distributed more densely at upper and lower ends of the body than in a middle portion of the body. In yet other embodiments, the fenestrated openings are distributed more densely in a middle portion of the body than at upper and lower ends of the body.

In a further embodiment, a vertebral implant for decompression and vertebral fusion is provided. The implant comprises an expandable interbody cage and an injection port. The expandable interbody cage comprises a superior endplate, an inferior endplate, and a body between the superior endplate and the inferior endplate. The body is defined by a wall extending at least partially between the superior endplate and the inferior endplate, wherein the wall includes a plurality of fenestrated openings. The injection port is configured for receiving a bone cement injector such that bone cement may be injected through the injection port into the body. The injection port may include a coupling mechanism for coupling to the bone cement injector/injection device. A seal may be provided for sealing the injection port such that injected bone cement cannot exit the injection port away from the interbody cage. The seal may comprise a one-way seal and/or an end cap. The fenestrated openings are configured to facilitate migration of injected bone cement out of the interbody cage, into disc space, and around the vertebral implant. In some embodiments, the interbody cage is expandable in an inferior/superior direction.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1a is a perspective view of a vertebral implant, in accordance with one embodiment.

FIG. 1b is a perspective view of a vertebral implant, in accordance with one embodiment.

FIG. 2a illustrates a top view of the vertebral implant of FIG. 1a.

FIG. 2b illustrates a top view of the vertebral implant of FIG. 2a.

FIG. 3a illustrates a front view of a telescoping, expanded interbody cage, in accordance with one embodiment.

FIG. 3b illustrates a side view of the implant of FIG. 3a.

FIG. 4a illustrates a top view of a rectangular interbody cage, in accordance with one embodiment.

FIG. 4b illustrates a side view of the rectangular interbody cage of FIG. 41.

### DETAILED DESCRIPTION

The present disclosure relates to a novel and advantageous implant for vertebral decompression and fusion that is not dependent upon new bone growth. In particular, a novel and advantageous implant for vertebral decompression and fusion that disperses bone cement in and around the disc space to create support between and fusion of adjacent vertebral bodies is provided. The implant includes an interbody cage and an injection port. The interbody cage comprises a superior endplate, an inferior endplate, and a body between the superior endplate and the inferior endplate. The interbody cage includes one or more fenestrated openings to facilitate migration of injected bone cement out of the interbody cage and into the disc space. The injection port is configured for coupling to a bone cement injector such that bone cement may be injected through the injection port into the body with the coupling being able to withstand the force required to inject relatively viscous bone cement. The injection port may include a seal for sealing the injection port.

In some embodiments, an implant is provided that allow a surgeon to perform a discectomy and decompression without requiring pedicle screws, bone forming graft, and/or biologics. In other embodiments, bone forming graft may be used in conjunction with the disclosed implant.

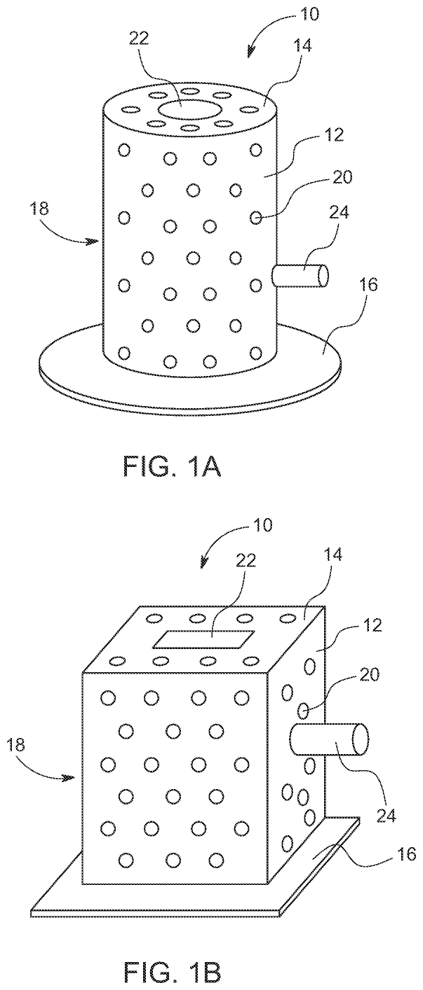

As shown in FIG. 1a, an implant 10 comprising an interbody cage 12 for facilitating bone cement dispersion is provided. In the embodiment of FIG. 1a, the interbody cage 12 has an approximately oval cross section. As shown, the interbody cage 12 may include a superior endplate 14, an inferior endplate 16, and a body 18 therebetween. The body may include a wall extending between the superior endplate 14 to the inferior endplate 16. In some embodiments, scaffolding may be provided within the body to support the endplates. The scaffolding may be provided within the wall and/or separate from the wall. The wall and/or the endplates may include fenestrated openings 20 and 22, or cement vent holes, for facilitating migration of the bone cement from the interbody cage into the disc space and around the implant. The fenestrated openings 20 and 22 may vary in shape and size or may be uniform. In the embodiment shown, at least one larger fenestrated opening 22 is provided on the superior endplate 14 and/or the inferior endplate 16. The wall and endplate(s) 14, 16 may be integrally formed or may be discrete and coupled.

A cement injection port 24 is provided through which bone cement may be injected into the interbody cage. The cement injection port 24 includes a coupling mechanism for coupling a cement injection device to the cement injection port. Such coupling mechanism provides a sufficiently strong connection between the cement injection device or injector and the injection port such that the connection can withstand the force and pressure needed to expel relatively viscous cement (in contrast to the relatively flowable bone graft that is typically used with a vertebral implant). A seal is provided with the cement injection port to substantially prevent cement from being released back through the cement injection port.

FIG. 1b illustrates an alternative embodiment of the implant 10 of FIG. 1a wherein the interbody cage has a rectangular cross section. A rectangular cross section may be useful with certain types of devices for placing the implant within the disc space. The orientation and sizing of various elements shown in FIGS. 1a and 1b are intended to provide guidance only and are not intended to be limited. Relative sizing of dimensions, for example, the widest or narrowest dimension may be varied and chosen as is well known by one skilled in the art. Further, relative placement of the injection port (discussed below) may be chosen based on specific use.

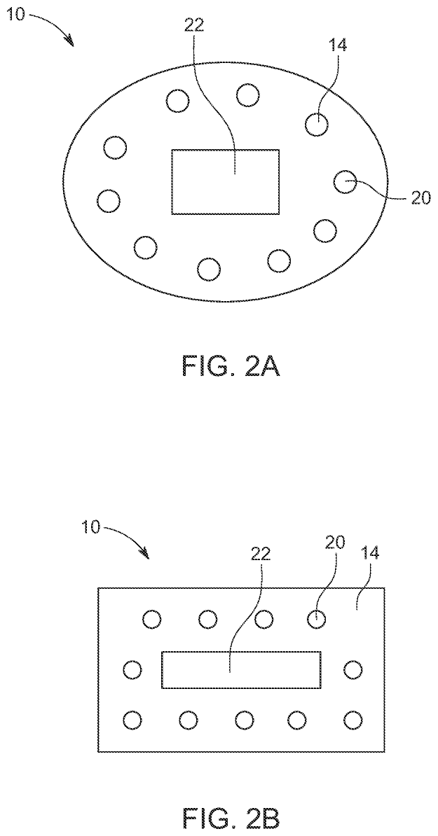

FIGS. 2a and 2b illustrate top views of the embodiments of FIGS. 1a and 1b, respectively without showing the inferior endplates.

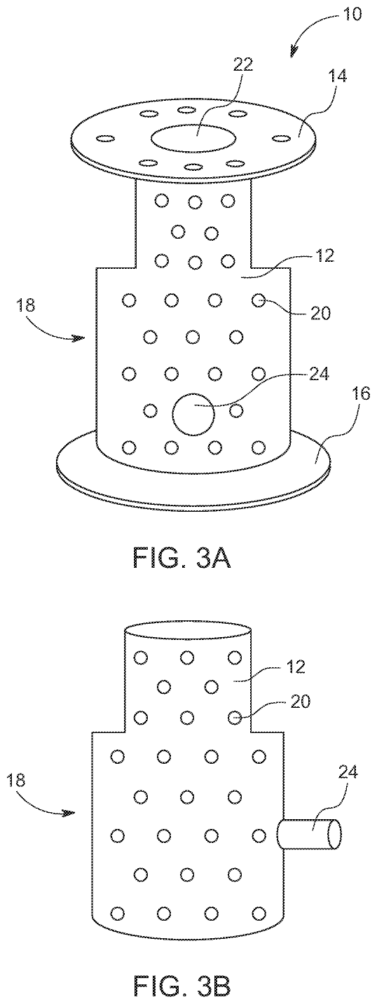

FIG. 3a illustrates a front view of an implant in accordance with one embodiment. FIG. 3b illustrates a side view of the implant of FIG. 3a with the endplates not being shown. FIGS. 3a and 3b illustrate an example of a telescoping, expanded interbody cage.

The vertebral implant disclosed herein is used with bone cement such as polymethyl methacrylate (PMMA). In some embodiments, a bone cement having a relatively low polymerization temperature and greater porosity may be used. For example, the bone cement may have added sucrose crystals, tricalcium phosphate, or other. Bone cement has a short cure time, on the order of 10 minutes or less. In contrast, bone grafting material frequently takes weeks to months to cure. By using bone cement, the cure time is reduced to in-operation curing and it is not necessary to use mechanical temporary fixation elements with the interbody cage. In some embodiments, a hybrid bone cement may be used. In other embodiments, a bone grafting material, such as allograft or autograft, or other bone growth promoting material may be used in combination with the bone cement. If cement and grafting material are used, grafting material may be injected into and through the interbody cage. After the grafting material has been injected, and migrated into the disc space, bone cement may be injected into the interbody cage. Alternatively, only bone cement may be injected into the interbody cage.

Bone cement is significantly more viscous and thick than grafting material. This means that the manner of injecting bone cement is generally different than the manner by which grafting material is typically injected into a vertebral implant. The vertebral implant includes an injection port to attach a bone cement injection apparatus to fill the cage, vertebral body cavity, and/or disc cavity with bone cement and, optionally, bone growth biologic material. More specifically, the vertebral implant may include a cement injection port for receiving an injection tool through which cement is injected into the interbody cage. In order to provide sufficient pressure to push the bone cement into the interbody cage, the injection tool may be removably coupled to the injection port. This may be, for example, via threading, a luer lock connection, a snap fit, or other coupling mechanism. The strength of the coupling is sufficient to withstand the pressure and force needed to push the bone cement into and through the vertebral implant.

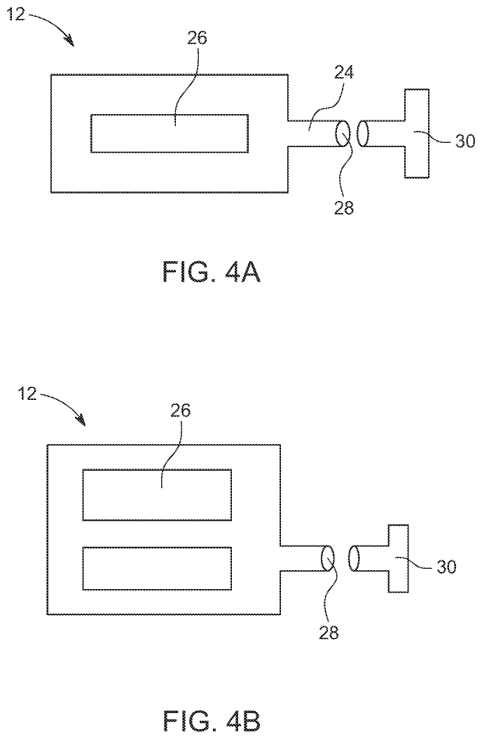

FIGS. 4a and 4b illustrate a side view and a top view of a vertebral implant having a rectangular interbody cage 12. In the embodiment of FIGS. 4a and 4b, one or more windows 26 are provided along surfaces of the interbody cage. As shown, a single window 26 is provided on each of the top and bottom surfaces and two windows 26 are provided on each of the side surfaces. The windows are a fenestration openings or vent holes for expulsion of cement, and optionally, graft material. It is to be appreciated that the windows shown are for example only and more or fewer windows may be provided on any surface and/or other fenestration opening may be provided on each surface.

An injection port 24 is provided through which bone cement may be injected into the interbody cage. The injection port 24 may include a coupling mechanism for coupling to a cement injection device sufficiently fixedly that the coupling can withstand the force necessary to inject relatively viscous bone cement. In one embodiment, the injection port may comprise a cannula wherein the cannula has a threaded inner shaft. A cement injection device, such as a cement injection mechanical gun having threads on an outer shaft, may be threaded into the injection port. Alternatively, a luer lock or other coupling mechanism may be used.

When injected into an interbody cage, material may tend to slide back out of the cage (via the injection port) when the injection device is removed. In the case of bone graft material, while this is not preferred, it is tolerable even if the bone graft material touches a nerve because the bone graft material will eventually dissolve. If cement leaks out through the injection port, it may come in contact with a nerve and lead to stenosis. The injection port thus is provided with a seal. The seal may be a separate seal that is placed over the injection port or may be integral to the injection port. For example, a one-way seal 28 may be provided with the injection port wherein the injection device is inserted through the one-way seal 28 to inject cement into the interbody cage. After injection, the injection device is withdrawn through the one-way seal and the seal keeps cement from exiting the interbody cage via the injection port. An end cap 30 may then be provided on or through the seal. Alternatively, an end cap may be placed over or in the injection port without use of a seal. In some embodiments, the end cap has an externally threaded extension that can be threaded into the internal threaded cannula of the injection port. In some embodiments, the end cap may comprise the seal.

In some embodiments, the interbody cage may be expandable in one or more directions. For example, the interbody cage may have an expandable height, and expandable width, and/or an expandable depth. In alternative embodiments, a static height cage may be used. In general, the width of the implant is larger than the depth of the implant. Trial sizes may be provided for determining appropriate size of the cage as implanted. The size of the implant may be adjusted before or after implantation.

Using an expandable cage, the cage may be inserted at a contracted height to reduce endplate disruption, nerve root retraction, and impaction forces. Upon insertion, the cage may be expanded to a desired height to restore sagittal balance and foraminal height, for example. In some embodiments, the cage is expandable in finite increments. For example, the contracted height of a cage may be 6 mm, 8 mm, or 10 mm and the expanded height of the cage may be 11 mm, 13 mm, or 15 mm. The cage may be configured to expand a single increment, such as a single 5 mm increment. Alternatively, the cage may be configured to expand up to a maximum height but at any increment, for example via a threaded expansion mechanism. In general, the cage may be expanded using an external tool after placement of the cage in the intervertebral space.

In some embodiments, the wall of ther interbody cage may be telescoping such that a height of the implant may be increased or decreased. In other embodiments, the wall may be non-fixedly attached to one of the inferior endplate or the superior endplate such that expansion of an interior scaffolding (separate from the wall) will cause an opening between the endplate and the wall. The opening provides an additional venting point for bone cement. If the wall is fixedly attached to the superior endplate and non-fixedly attached to the inferior endplate, expansion of the scaffolding will lift the wall from the inferior endplate and the venting opening will be provided at a lower portion of the cage. If the wall is fixedly attached to the inferior endplate and non-fixedly attached to the superior endplate, expansion of the scaffolding will lift superior end plate from the wall and the venting opening will be provided at an upper portion of the cage.

The endplates and the wall may include one or more fenestrated openings, or vent holes, through which cement can migrate into the disc space and around the implant. Any suitable shape and size of vent holes may be used. For example, in the embodiments of FIGS. 1a and 2a, a plurality of round vent holes, each being approximately 1-2 mm are provided. The vent holes may be provided in a regular distribution. In alternative embodiments, more vent holes may be provided towards upper and lower ends than in the middle of a height of the cage, or more vent holes may be provided towards the middle of a height of the cage than towards upper and lower ends. In general, facilitating migration of the cement through the endplates and also through the body allows for a more complete fixation of the implant in the disc space. In the embodiment of FIGS. 4a and 4b, rectangular vent holes are provided.

In the embodiment shown in FIG. 1a, the cage has an inferior endplate with an increased width versus the body of the cage and the superior endplate. In alternative embodiments, the superior and inferior endplates may have the same width as one another but an increased width versus the body of the cage. In yet other embodiments, the superior and inferior endplates and the body of the cage may have the same width. The width of the endplate(s) may be based on the height of the cage. For example, a cage having a 6 mm height may have an 8 mm width endplate, a cage having an 8 mm height may have a 10 mm width endplate, and a cage having a 10 mm height may have a 12 mm width endplate.

In general, the interbody cage may have any suitable depth, width, and height footprints and may be expandable in any of these directions. Further, in some embodiments a series of differently sized implants may be provided from which a surgeon can select the appropriate size for an individual patient. In some embodiments, the injection port may be provided on a different side of the implant.

The implant may be used with instruments for creating a cavity between two adjoining vertebral bodies. The implant allows injection of either bone cement, optionally with a bone growth promoting material, wherein the bone cement yields near instant fixation and stabilization of the two vertebral bodies. This near instant stability of the two or more vertebral bodies reduces or eliminates the need for expensive and damaging hardware constructs that only are designed for temporary fixation until the bone grows into a fusion.

The implant may comprise polyetheretherketone (peek), titanium, or other suitable material. As discussed above, the implant may be expandable in height, depth, and/or width. In some embodiments, the interbody cage may be expandable in height only (referred to as inferior/superior expansion). The interbody thus may be placed in the intervertebral space and expanded to a desired height before injection of bone cement.

In some embodiments, an existing interbody cage may be modified or retrofit to accept bone cement and to facilitate migration of the bone cement from the cage into the disc space and around the interbody cage. Such modification may comprise an adapter for the injection port wherein the adapter can receive and couple to a cement injection device such that it can withstand the force required to inject viscous cement. The adapter may further include an end cap to prevent leaking of the cement from the injection port.

The implant and technique provided herein provides nearly instant bone fusion and stability via intervertebral void creation, vertebral bone void creation (inside the vertebral body via the endplates of the vertebral body), and cement insertion with the indication for instantaneous bone fusion and stability following interbody discectomy and decompression. In some embodiments, pedicle screws or other stabilizing structure may be used with the interbody cage.

In one embodiment, a method for vertebral decompression and fusion is provided. The method includes performing a discectomy and laminectomy, inserting an interbody cage into the created void space, and inserting cement (and or other bone morphing protein) into the interbody cage such that the cement migrates out of the interbody cage into the created intervertebral void and vertebral bone void. The cement cures in 5-10 minutes and provided near instant structural support between the vertebral bodies, decompressing the nerve and fusing the vertebral bodies.

The implant and method provided herein use bone cement in the place of scaffolding and surgical components for vertebral decompression fusion, in accordance with one embodiment. Disc and soft tissues around the nerves are removed to create a void. An interbody cage including a cement injection port and cement vent holes is placed in the created void. Cement is injected and migrates around the disc space and vertebral body. The amount of cement injected varies based on the size of the adjacent vertebral bodies and the void space. The bone cement provides a permanent structural support and does not rely on bone growth, although bone growth promoting materials may additionally by used. In some embodiments, a hybrid approach including bone cement as well as traditional structural elements such as screws and rods may be provided.

While cement is discussed herein, it is to be appreciated that bone growth promoting material may be used in conjunction with bone cement. Further, other types of quick-curing materials that will provide structural support between the vertebral bodies may be used.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A system for decompression and vertebral fusion, the system comprising:
a vertebral implant:
an interbody cage comprising:
a superior endplate;
an inferior endplate;
a body between the superior endplate and the inferior endplate; and
an injection port configured for receiving a bone cement injector such that bone cement may be injected through the injection port into the body;
wherein the body is configured to facilitate migration of injected bone cement out of the interbody cage, into disc space, and around the vertebral implant; and
bone cement for injection into the interbody cage and migration out of the interbody cage, into disc space, and around the vertebral implant.

2. The system of claim 1, wherein the body is defined by a wall extending at least partially between the superior endplate and the inferior endplate.

3. The system of claim 2, wherein the wall includes a plurality of fenestrated openings for facilitating the migration of injected bone cement.

4. The system of claim 1, wherein the wall is telescoping and a height of the interbody cage may be adjusted by telescoping the wall.

5. The system of claim 4, wherein the height is adjusted using an adjustment tool.

6. The system of claim 1, wherein the injection port includes a coupling mechanism for coupling to the bone cement injector.

7. The system of claim 6, wherein the coupling mechanism is threading within an inner cannula of the injection port.

8. The system of claim 6, wherein the injection port further includes a seal for sealing the injection port such that injected bone cement cannot exit the injection port away from the interbody cage.

9. The system of claim 1, wherein at least one of the superior endplate and the inferior endplate includes fenestrated openings for facilitating the migration of injected bone cement.

10. The system of claim 1, wherein the superior endplate has a width and the inferior endplate has a width, and wherein the width of the inferior endplate exceeds the width of the superior endplate.

11. The system of claim 1, wherein the superior endplate has a width and the inferior endplate has a width, and wherein the width of the inferior endplate matches the width of the superior endplate.

12. The system of claim 1, wherein the bone cement comprises a polymethyl methacrylate modified to have a low polymerization temperature and high porosity compare to unmodified polymethyl methacrylate.

13. A vertebral implant for decompression and vertebral fusion, the implant comprising:
an interbody cage comprising:
a superior endplate;
an inferior endplate;
a body between the superior endplate and the inferior endplate, wherein the body is defined by a wall extending at least partially between the superior endplate and the inferior endplate, and wherein the wall includes a plurality of fenestrated openings; and
an injection port configured for receiving a bone cement injector such that bone cement may be injected through the injection port into the body, wherein the injection port includes a coupling mechanism for coupling to the bone cement injector and a seal for sealing the injection port such that injected bone cement cannot exit the injection port away from the interbody cage;
wherein the fenestrated openings are configured to facilitate migration of injected bone cement out of the interbody cage, into disc space, and around the vertebral implant.

14. The vertebral implant of claim 13, wherein the fenestrated openings are evenly distributed over the wall.

15. The vertebral implant of claim 14, wherein the fenestrated opening are distributed more densely at upper and lower ends of the body than in a middle portion of the body.

16. The vertebral implant of claim 14, wherein the fenestrated openings are distributed more densely in a middle portion of the body than at upper and lower ends of the body.

17. The vertebral implant of claim 14, wherein at least one of the superior endplate and the inferior endplate includes a plurality of fenestrated openings to facilitate the migration of injected bone out of the interbody cage, into disc space, and around the vertebral implant.

18. A vertebral implant for decompression and vertebral fusion, the implant comprising:
an expandable interbody cage comprising:
a superior endplate;
an inferior endplate;
a body between the superior endplate and the inferior endplate, wherein the body is defined by a wall extending at least partially between the superior endplate and the inferior endplate, wherein the wall includes a plurality of fenestrated openings;
an injection port configured for receiving a bone cement injector such that bone cement may be injected through the injection port into the body, wherein the injection port includes a coupling mechanism for coupling to the bone cement injector and a seal for sealing the injection port such that injected bone cement cannot exit the injection port away from the interbody cage;
wherein the fenestrated openings are configured to facilitate migration of injected bone cement out of the interbody cage, into disc space, and around the vertebral implant.

19. The vertebral implant of claim 18, wherein the interbody cage is expandable in an inferior/superior direction.

20. The vertebral implant of claim 19, wherein the body is telescoping.
